(19) United States
(12) Patent Application Publication
(10) Pub. No.: US 2009/0317372 A1
(43) Pub. Date: Dec. 24, 2009

(54) SMALL MOLECULES FOR THE REDUCTION OF HIGH BLOOD GLUCOSE LEVEL

(76) Inventor: Zoltan Kiss, Austin, MN (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/394,789

(22) Filed: Feb. 27, 2009

Related U.S. Application Data
(60) Provisional application No. 61/074,329, filed on Jun. 20, 2008.

(51) Int. Cl. A61K 38/46; A61K 31/382; A61K 38/40; A61P 3/10

(52) U.S. Cl. 424/94.6; 514/437; 514/8

(57) ABSTRACT

Embodiments of the present invention include the in vivo use of a family of heterocyclic compounds containing a quaternary ammonium group as exemplified by the thioxanthone and thioxanthene compounds [3-(3,4-dimethyl-9-oxo-9H-thioxanthen-2-yloxy)-2-hydroxypropyl]trimethylammonium chloride, or CCcompound1, N,N,-diethyl-N-methyl-2-[9-oxo-9H-thioxanthen-2-yl)methoxy]ethanaminium iodide, or CCcompound3, and N,N,N-trimethyl-3-(9H-thioxanthen-9-ylidene)-propane-1-aminium iodide, or CCcompound19 to reduce higher than normal blood glucose level within or close to the normal range in subjects with insulin resistance, hyperglycemia, and diabetes thereby also reducing or preventing associated diseases, complications, and pathological states.

### CROSS REFERENCE TO RELATED APPLICATION

This application claims the benefit of provisional application Ser. No. 61/074,329 filed Jun. 20, 2008, titled “Small Molecules to Normalize Pathological Levels of Blood Glucose” which is herein incorporated by reference in its entirety.

### TECHNICAL FIELD

The invention generally relates to a family of heterocyclic compounds containing a quaternary ammonium group as exemplified by the thioxanthone and thioxanthene compounds [3-(3,4-dimethyl-9-oxo-9H-thioxanthen-2-yloxy)-2-hydroxypropyl]trimethylammonium chloride, or CCcompound1, N,N,-diethyl-N-methyl-2-[9-oxo-9H-thioxanthen-2-yl)methoxy]ethanaminium iodide, or CCcompound3, and N,N,N-trimethyl-3-(9H-thioxanthen-9-ylidene)-propane-1-aminium iodide, or CCcompound19, to reduce higher than normal blood glucose level into or closer to the normal physiological range without causing hypoglycemia as well as reduce, delay or prevent diseases, complications, and pathological states associated with hyperglycemia and diabetes.

### BACKGROUND

According to a recent estimate, about ten percent of Americans will develop type 2 diabetes during their lifetime. This type of diabetes is preceded by impaired glucose tolerance (IGT) that >20 percent of Americans will develop. IGT results from decreased uptake and metabolism of glucose by target tissues and is the consequence of insulin resistance and dyslipidemia. IGT is defined as a serum glucose concentration between 140-199 mg/dl 2 hours after a 75-g glucose load, while diabetes is defined as a 2 hours value of 200 mg/dl or higher [Singleton, J. R., Smith, A. G., Russell, J. W. and Feldman, E. L. (2003) Microvascular complications of impaired glucose tolerance. Diabetes 52, 2867-2873].

Proper functioning and survival of each organ requires continuous supply of glucose. However, blood glucose level needs to be maintained in a relatively narrow range around 5 mM, because hypoglycemia can lead to cell death while chronic hyperglycemia causes organ damage that can result in cardiomyopathy, cardiovascular disease, a variety of neuropathies, retinopathy, nephropathy and other diseases.

A major regulator of blood glucose is insulin which, following a meal, is secreted from the pancreatic islet β-cells normally only at needed amounts. The primary targets of insulin are skeletal and cardiac muscle, adipose tissue and liver. The rate-limiting step in glucose utilization and storage is its uptake into the muscle and fat cells by specific transporters. If the concentration of glucose is lowered below 5 mM, pancreatic α-cells secrete glucagon which increases gluconeogenesis and glycolysis in the liver thereby re-adjusting the normal blood glucose level. Normally, insulin inhibits gluconeogenesis and glycolysis in the liver.

If the target tissues do not respond to respective stimulatory and inhibitory effects of insulin to sufficient extents this results in IGT and hyperglycemia. Since normally insulin inhibits hormone sensitive lipase-mediated lypolysis in adipocytes, insulin resistance of the adipose tissue results in increased formation and release of fatty acids into the circulation which further reduces the insulin effects in the muscle. Obesity may result in the amount of free fatty acids in the circulation being high. Therefore, insulin resistance often develops in obese subjects eventually leading to hyperglycemia and diabetes [Erdman, J., Kallabis, B., Oppel, U., Sypchenko, O., Wagenpfell, S. and Schusdziarra, V. (2008) Development of hyperinsulinemia and insulin resistance during the early stage of weight gain. Am. J. Physiol. Endocrinol. Metab. 294, E568-E575]. It is estimated that about 60% of type 2 diabetes cases are due to obesity while the rest of cases have other causes. For example, cachexia, sepsis, pregnancy, starvation, burn trauma, metabolic syndrome, and acromegaly are risk factors for hyperglycemia and diabetes. Also, patients with cancer often exhibit insulin resistance and hyperglycemia [Lundholm, K., Holm, G. and Schersten, T. (1978) Insulin resistance in patients with cancer. Cancer Res. 38, 4665-4670]. The reverse is also true: many studies indicate that insulin resistance and hyperinsulinemia are risk factors for various cancers [Giovannucci, E. (2005) The role of insulin resistance and hyperinsulinemia in cancer causation. Curr. Med. Chem.-Immun. Endoc. & Metab. Agents 5, 53-60; Flood, A., Mai, V., Pfeiffer, R., Kahle, L., Relaley, A. T. Lanza, E. and Schatzkin, A. (2007) Elevated serum concentrations of insulin and glucose increase risk of recurrent colorectal adenomas. Gastroenterology 133, 1423-1429; Stattin, P., Bjor, O., Ferrari, P., Lukanova, A., Lenner, P., Lindahl, B., Hallmans, G. and Kaaks, R. (2007) Prospective study of hyperglycemia and cancer risk. Diabetes Care 30, 561-567].

A special case is the relationship between diabetes and pancreatic cancer. Recent studies indicate that pancreatic cancer not only causes diabetes via triggering dysfunction of islet cells, but also causes insulin resistance; furthermore, diabetes is a risk factor for pancreatic cancer [Wang, F., Gupta, S. and Holly, E. A. (2006) Diabetes mellitus and pancreatic cancer in a population-based case-control study in the San Francisco Bay Area, California. Cancer Epidemiol. Biomerkers Prev. 15, 1458-1463; Chari, S. T., Leibson, C. L., Rabe, K. G., Timmons, L. J., Ransom, J., De Andrade, M. and Petersen, G. M. (2008) Pancreatic cancer-associated diabetes mellitus: Prevalence and temporal association with diagnosis of cancer. Gastroenterology 134, 95-101; Pannala, R., Leirness, J. B., Bamlet, W. R., Basu, A., Petersen, G. M. and Chari, S. T. (2008) Prevalence and clinical profile of pancreatic cancer-associated diabetes mellitus. Gastroenterology 134, 981-987].

Some treatments can also lead to insulin resistance; for example, such drugs include HIV-protease inhibitors [Carper, M. J., Cade, W. T., Cam, M., Zhang, S., Shalev, A., Yarashenski, K. E. and Ramanadham, S. (2007) HIV-protease inhibitors induce expression of suppressor of cytokine signaling-1 in insulin-sensitive tissues and promote insulin resistance and type 2 diabetes mellitus. Am. J. Physiol. Endocrinol. Metab. 294, E558-E567].

Diabetes is a potentially very dangerous disease because it is associated with markedly increased incidence of coronary, cerebral, and peripheral artery disease. As a result, patients with diabetes have a much higher risk of myocardial infarction, stroke, limb-amputation, renal failure, diabetic wounds, or blindness.

Generally, there is also an association between hyperglycemia/diabetes and mortality of critically ill patients [Krinsley, J. S. (2003) Association between hyperglycemia and increased hospital mortality in a heterogeneous population of critically ill patients. Mayo Clin. Proc. 78, 1471-1478] including cancer patients [Batty, G. D., Shipley, M. J., Marmot, M. and Smith, G. D. (2004) Diabetes status and post-load plasma glucose concentration in relation to site-specific cancer mortality: findings from the original Whitehall study. Cancer Causes Contr. 15, 873-881; Borugian, M. J., Sheps, S. B., Kim-Sing, C., Patten, C. V., Potter, J. D., Dunn, B., Gallagher, R. P. and Hislop, T. G. (2004) Insulin, macronutrient intake and physical activity: Are potential indicators of insulin resistance associated with mortality from breast cancer? Cancer Epidemiol. Biomarkers Prev. 13, 1163-1172].

In Type 2 diabetes, an aggressive control of hyperglycemia by medication is essential; otherwise this disease will progress into the even more dangerous Type 1 diabetes. Several drugs in five major categories, each acting by a different mechanism and none fully effective in itself, are available for this purpose.

(i) Insulin secretogogues, including sulphonylureas (e.g., Tolbutamide, Glimepiride, Glyburide, Glipizide, Tolazamide, Acetohexamine, Chlorpropamide), meglitinides (e.g., Nateglidine and Repaglinide), incretin hormones (glucagon-like peptide and glucose-dependent insulinotropic peptide as well as their analogs), and inhibitors of dipeptidyl peptidase-4 (Sitagliptin) enhance secretion of insulin by acting on the pancreatic β-cells. While these therapies can decrease blood glucose level, they may have limited efficacy and tolerability. In addition, they usually cause weight gain and may induce hypoglycemia. Finally, patients often become refractory to these treatments.

(ii) Biguanides (e.g., Metformin or Glucophage) are thought to act in part by stimulating AMP kinase (AMPK) activity thereby decreasing glucose production in the liver. Biguanides often cause gastrointestinal disturbances and lactic acidosis, which may limit their use.

(iii) Inhibitors of α-glucosidase (e.g., Acarbose, Miglitol) decrease absorption of glucose from the intestine. These agents also often cause gastrointestinal disturbances.

(iv) Thiazolidinediones (e.g., Pioglitazone, Rosiglitazone) act on a specific receptor (peroxisome proliferator-activated receptor-gamma or PPARγ). They primarily regulate lipid metabolism and thus enhance the response of fat and other tissues to the actions of insulin. Recent studies indicate that they also enhance survival of β-cells and may directly enhance glucose transport in the skeletal muscle. On the negative side, frequent use of these drugs may lead to weight gain and may induce edema and anemia. Some studies also hint that this class of drugs may enhance the number of cardiovascular events.

(v) Insulin is used in more severe cases, either alone or in combination with the above agents. The real danger with insulin is that it may cause hypoglycemia. It also increases weight gain which, paradoxically further reduces insulin sensitivity of peripheral tissues.

All these medications are given to the patient, alone or in various combinations (Metaglip, Glipizide+Metformin; Avandamet, Rosiglitazone+Metformin; Glucovance, Glyburide+Metformin; ActoPlus, Pioglitazone+Metformin; Avandaryl, Pioglitazone+Glimepiride; Janumet, Sitagliptin+Metformin; Duetact, Pioglitazone and Metformin) often two or three times a day.

Each of these agents has some side effect such as weight gain. Even more importantly, almost all agents become less efficient after prolonged treatments. For these reasons, new approaches to control Type 2 diabetes are needed.

### SUMMARY OF THE INVENTION

The present invention relates to the use of heterocyclic compounds containing a quaternary ammonium group as exemplified by the thioxanthone and thioxanthene compounds [3-(3,4-dimethyl-9-oxo-9H-thioxanthen-2-yloxy)-2-hydroxypropyl]trimethylammonium chloride, or CCcompound1, N,N,-diethyl-N-methyl-2-[9-oxo-9H-thioxanthen-2-yl)methoxy]ethanaminium iodide, or CCcompound3, and N,N,N-trimethyl-3-(9H-thioxanthen-9-ylidene)-propane-1-aminium iodide, or CCcompound19 to reduce higher than normal blood glucose level within, or closer to, the normal range in subjects with insulin resistance, hyperglycemia and diabetes.

Several CC compounds significantly decreased blood glucose level in glucose tolerance tests performed with non-diabetic mice and rats as well as obese diabetic mice. The effects were particularly pronounced during the first 2 hours of treatment. CC compounds also lowered blood glucose level in normally fed diabetic mice particularly during the first hour of the treatment. Thus, a suitable CC compound can bring pathologically high blood glucose levels within, or closer to, the normal physiological range.

In one embodiment, the CC compound is administered to a subject with insulin resistance, hyperglycemia or type 2 diabetes to induce rapid reduction in the abnormally high blood glucose level.

In another embodiment a CC compound is administered to a subject with insulin resistance, hyperglycemia or type 2 diabetes shortly prior to a meal to prevent large excursions in the blood glucose level during and after the meal.

In a further embodiment, a CC compound is administered to a subject whose insulin resistance, hyperglycemia or diabetes results from one of the following conditions; cachexia, cancer, sepsis, pregnancy, starvation, burn trauma, metabolic syndrome, obesity, or acromegaly.

A suitable CC compound may be administered alone or along with an oral or injectable antidiabetic agent and/or any standard treatment that is employed to treat cachexia, cancer, sepsis, pregnancy, starvation, burn trauma, metabolic syndrome, diabetic wounds, obesity, or acromegaly, to reduce or prevent hyperglycemia.

In a certain embodiment, the invention provides a treatment regimen for the treatment of a mammal with insulin resistance, hyperglycemia or type 2 diabetes comprising periodically administering a therapeutically effective amount of a suitable CC compound alone or together with another diabetes treatment or any treatment indicated for the above conditions.

In another embodiment, the treatment regimen for the treatment of a mammal with insulin resistance, hyperglycemia or type 2 diabetes is provided to improve or prevent complications resulting from elevated blood glucose level including increased mortality from critical illness and macrovascular as well as microvascular or other tissue injury-related events leading to cardiomyopathy, cardiovascular disease, various neuropathies, retinopathy, nephropathy, stroke, and diabetic wounds.

In yet another embodiment, the treatment regimen is provided to reduce or overcome insulin resistance and hyperglycemia in cancer patients.

In a further embodiment, the treatment regimen is provided to reduce or overcome insulin resistance and high blood glucose level in obese subjects.

In an additional embodiment, the invention provides for the use of a CC compound in the manufacture of a composition useful for the normalization of pathologically high levels of blood glucose.

In some embodiments, the mammal is administered a therapeutically effective amount of a suitable CC compound such as CCcompound1, CCcompound3 or CCcompound19. The term “therapeutically effective amount” is used in this application to mean a dose that significantly reduces high blood glucose level without causing hypoglycemia. The term “pathologically high blood glucose level” means that either an untreated subject has a blood glucose higher than 6 mM, or during a meal or a glucose tolerance test elevated blood glucose returns to the normal level only slowly compared to healthy subjects, for example due to resistance of peripheral tissues to insulin and/or insufficient secretion of insulin. The term “reduce or overcome insulin resistance” does not necessarily means that the CC compound enhances either the insulin effect or insulin secretion. A CC compound may reduce or overcome insulin resistance at least in part merely by acting via an insulin-independent mechanism thereby lessening the burden on the insulin-dependent system.

In some embodiments, patients that have abnormally high blood glucose levels are subjected to any of the treatments described herein. For example, an indication for the treatments described herein may be a blood glucose level that is higher than about 6 mM, higher than about 7 mM, higher than about 8 mM, higher than about 10 mM, higher than about 15 mM, or higher than about 20 mM. Another indication for the treatments described herein may be that a subject has a history of blood glucose levels being above any of these levels and is about to undergo an event that may increase blood glucose levels (for example, the subject is about to have a large meal). Additional examples of such methods of treatment are described below.

### DETAILED DESCRIPTION OF THE INVENTION

### Active Components

The compounds used in the application, collectively termed “CC compounds”, contain a heterocyclic moiety to which a quaternary ammonium-containing moiety is attached at one or more of the following positions; the R₂, R₁₀, V and/or Y of the heterocyclic moiety represented by the formula:

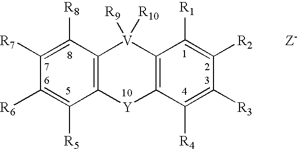

wherein R1 and R3-8 are independently hydrogen, C1-C26 straight, branched or cyclic alkanes or alkenes, aromatic hydrocarbons, alcohols, ethers, aldehydes, ketones, carboxylic acids, amines, amides, nitriles, or five- and/or six-membered heterocyclic moieties; wherein R9 and R10 considered together are ═O or ═CH-L-N⁺(R11, R12, R13) or wherein R9 and R10 considered independently are —OH or -L-N⁺(R11, R12, R13);

wherein R2 is represented by the formula: —X or —X′-L-N⁺(R 11, R12, R13)Z⁻ or -L-N+(R11, R12, R13)Z⁻;

wherein V is —S—, —Se—, —C—, —O— or —N;

wherein Y is —S—, —Se—, —C—, —O— or —N;

wherein -L-N⁺(R11, R12, R13) can be linked to V or Y if V or Y is —N or can be linked to V and Y if V and Y are both —N;

wherein X is CH3 or Hydrogen or —OH;

wherein —X′ is —CH2-, —OCH2-, —CH20-, —SCH2- or —CH2S—;

wherein L is a C1-C4 straight alkane, alkene, thiol, ether, alcohol, or amine;

wherein R11, R12 and R13 are independently Hydrogen, C1-C4 straight alkanes, alkenes, thiols, amines, ethers or alcohols; and

wherein Z- is Cl⁻, Br⁻ or I⁻.

One embodiment of these compounds is [3-(3,4-Dimethyl-9-oxo-9H-thioxanthen-2-yloxy)-2-hydroxypropyl]trimethyl-ammonium chloride, or CCcompound1. Two other embodiments of these compounds are N,N-diethyl-N-methyl-2-[(9-oxo-9H-thioxanthen-2-yl)methoxy]-ethanaminium iodide or CCcompound3, and N,N,N-trimethyl-3-(9H-thioxanthen-9-ylidene)-propane-1-aminium iodide, or CCcompound19. Exemplary methods of synthesizing representative CCcompounds are described in U.S. patent application Ser. No. 11/458,502, filed Jul. 19, 2006, entitled “Compounds and compositions to control abnormal cell growth”; inventor: Zoltan Kiss, which is incorporated herein by reference in its entirety.

The Examples described below show that the CC compounds possess glucose lowering properties.

Table 1 shows a representative list of CC compounds.

TABLE 1
A Representative List of CC compounds Used in the Invention.

| Trivial name | Chemical name | Structure |
|---|---|---|
| CCcompound 1 | [3-(3,4-Dimethyl-9-oxo- 9H-thioxanthen-2-yloxy)- 2- hydroxypropyl]trimethyl- ammonium chloride | 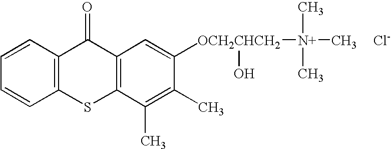 |
| CCcompound 2 | N,N,N-Trimethyl-2-[(9- oxo-9H-thioxanthen-2- yl)methoxy]- ethanaminium iodide | 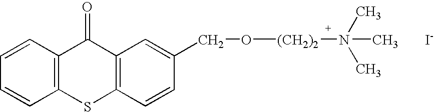 |
| CCcompound 3 | N,N-Diethyl-N-methyl-2- [9-oxo-9H-thioxanthen-2- yl)methoxy]- ethanaminium iodide | 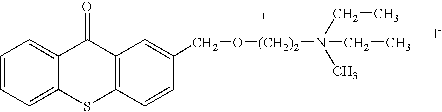 |
| CCcompound 4 | N,N,N-Triethyl-2-[(9-oxo- 9H-thioxanthen-2- yl)methoxy]- ethanaminium iodide | 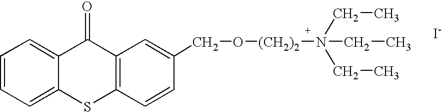 |
| CCcompound 5 | N-Ethyl-N,N-dimethyl-2- [(9-oxo-9H-thioxanthen-2- yl)methoxy]- ethanaminium iodide | 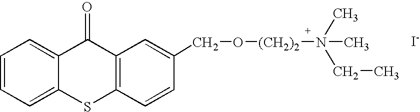 |
| CCcompound 6 | 2-{[2- (Diethylamino)ethoxy]me- thyl}-9H-thioxanthen-9- one hydrochloride | 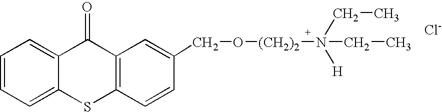 |
| CCcompound 7 | N,N,N-Trimethyl-3-[(9- oxo-9H-thioxanthen-2- yl)methoxy]-propane-1- aminium iodide | 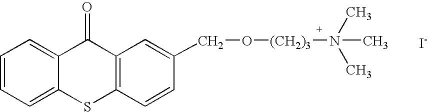 |
| CCcompound 8 | 2-{[3- (Dimethylamino)propoxy] methyl}-9H-thioxanthen- 9-one hydrochloride | 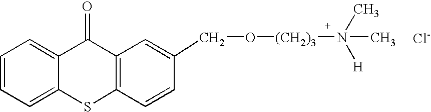 |
| CCcompound 9 | N,N,N-Triethyl-3-[(9-oxo- 9H-thioxanthen-2- yl)methoxy]-propane-1- aminium iodide | 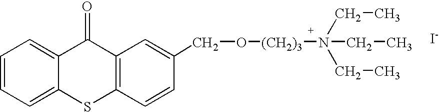 |
| CCcompound 10 | N,N-Diethyl-N-methyl-3- [(9-oxo-9H-thioxanthen- 2-yl)methoxy]-propane- 1-aminium iodide | 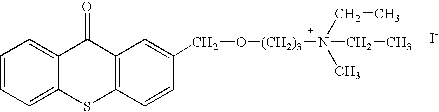 |
| CCcompound 11 | N,N-Dimethyl-N-ethyl-3- [(9-oxo-9H-thioxanthen- 2-yl)methoxy]-propane- 1-aminium iodide | 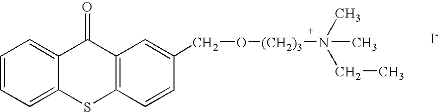 |
| CCcompound 12 | 2-{[3- (Diethylamino)propoxy] methyl}-9H-thioxanthen-9- one hydrochloride | 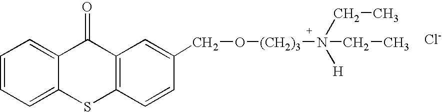 |
| CCcompound 13 | 2-Hydroxy-N,N- dimethyl-N-[(9-oxo-9H- thioxanthen-2-yl)methyl]- ethanaminium bromide | 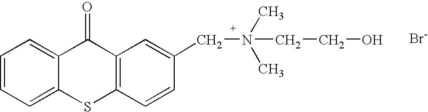 |
| CCcompound 14 | 2-Hydroxy-N,N-Diethyl- N-[(9-oxo-9H- thioxanthen-2-yl)methyl]- ethanaminium bromide | 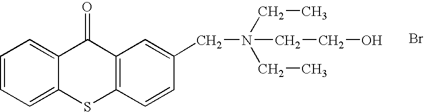 |
| CCcompound 15 | 3-Hydroxy-N,N-dimethyl- N-[(9-oxo-9H- thioxanthen-2- yl)methyl]propane-1- aminium bromide | 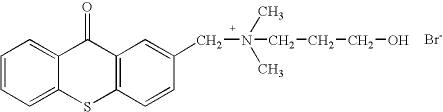 |
| CCcompound 16 | 3-Hydroxy-N,N-diethyl- N-[(9-oxo-9H- thioxanthen-2-yl)methyl]- propane-1-aminium bromide | 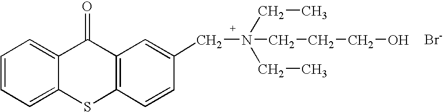 |
| CCcompound 17 | 3-(9-hydroxy-9H- thioxanthen-9-yl)-N,N,N- trimethyl-propane-1- aminium iodide | 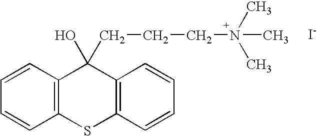 |
| CCcompound 18 | 3-(9-hydroxy-9H- selenoxanthen-9-yl)- N,N,N-trimethyl-propane- 1-aminium iodide | 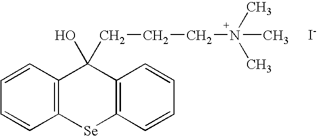 |
| CCcompound 19 | N,N,N-trimethyl-3-(9H- thioxanthen-9-ylidene)- propane-1-aminium iodide | 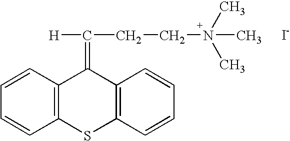 |
| CCcompound 20 | N,N,N-trimethyl-3-(9H- selenoxanthen-9-ylidene)- propane-1-aminium iodide | 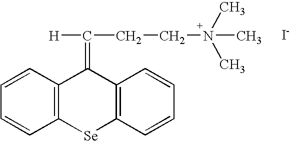 |
| CCcompound 21 | N,N,N-trimethyl-3-(2- methyl-9H-thioxanthen-9- ylidene)-propane-1- aminium iodide | 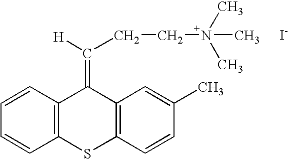 |
| CCcompound 22 | N,N-Dimethyl-N-ethyl-3- (2-methyl-9H- thioxanthen-9-ylidene)- propane-1-aminium iodide | 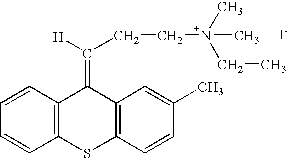 |
| CCcompound 23 | N,N-Diethyl-N-methyl-3- (2-methyl-9H- thioxanthen-9-ylidene)- propane-1-aminium iodide | 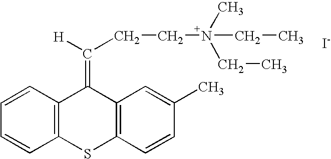 |
| CCcompound 24 | N,N-Dimethyl-N-allyl-3- (2-methyl-9H- thioxanthen-9-ylidene)- propane-1-aminium bromide | 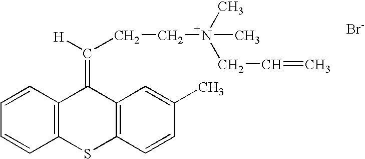 |
| CCcompound 25 | N,N,N-Triethyl-3-(2- methyl-9H-thioxanthen-9- ylidene)-propane-1- aminium iodide | 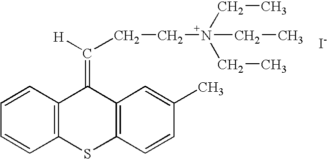 |
| CCcompound 26 | N,N-Diethyl-N-allyl-3-(2- methyl-9H-thioxanthen-9- ylidene)-propane-1- aminium bromide | 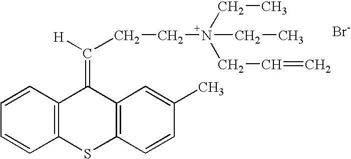 |

### Methods of Treatments

CC compounds are used in this invention to reduce higher than normal level of blood glucose in subjects with hyperglycemia or diabetes by overcoming insulin resistance and/or reduced insulin secretion. Consequentially, they are also suitable to prevent or reduce complications resulting from insulin resistance, hyperglycemia and diabetes including increased mortality from critical illness and macrovascular as well as microvascular or other tissue injury-related events leading to cardiomyopathy, cardiovascular disease, various neuropathies, retinopathy, nephropathy, stroke, and diabetic wounds.

The CC compounds are well soluble in water as well as in dimethylsulfoxide. Accordingly, oral application is one of the major administration routes to deliver a CC compound. In one embodiment of the invention, the CC compound is in the form of a tablet, gel capsule, a liquid, or the like. In each case, the CC compound is mixed with one or more carriers chosen by one having ordinary skill in the art to best suit the goal of treatment. In addition to the active compounds, the tablet or gel capsule may contain any component that is presently used in the pharmaceutical field to ensure firmness, stability, solubility and appropriate taste. In some embodiments, additional components of the tablet or gel will be chemically inert, i.e., it will not participate in a chemical reaction with the CC compound or the additives.

CC compounds may also be applied via intravenous, intraarterial, intraportal, intradermal, intraperitoneal, subcutaneous, intra-tissue or intramuscular delivery routes. In some embodiments, the CC compound may be delivered via infusion over a period of time or by using an osmotic minipump inserted under the skin for controlled release. The injectable solution may be prepared by dissolving or dispersing a suitable preparation of the CC compound in water or water-based carrier such as 0.9% NaCl (physiological saline) or phosphate buffered saline. Alternatively, the CC compound may be dissolved first in dimethylsulfoxide and then diluted (100-400-fold dilution) in a physiologically compatible carrier using conventional methods. As an example only, a suitable composition for the practice in the method comprises a CC compound in a 0.9% physiological saline solution to yield a total CC compound concentration of between about 0.1-g/ml and about 25.0-g/ml, between about 1.0-g/ml and about 10.0-g/ml, about 0.1-g/ml, about 10.0-g/ml, or about 25.0-g/ml.

A suitable dosage for oral or injection administration may be calculated in milligrams or grams of the active agent(s) per square meter of body surface area for the subject. In one embodiment, the therapeutically effective amount of CC compound is administered orally at a dose between 100-mg to 2,000-mg, or between 200-mg to 1,000-mg, per m² body surface of the mammal. In another embodiment, the CC compound is administered by an injection method at a dose of 50-mg to 1,000-mg, or between 100-mg to 500-mg, per m² body surface of the mammal.

The amount of the CC compound may vary depending on the method of application. For example, in case of intravenous application the required amount may approach the lower limit, while in case of subcutaneous application the required amount may be closer to the upper limit. Also, if oral application is repeated several times a day, the dose may be lowered.

Application of the CC compound orally or by an injection method may be repeated as many times as needed to achieve a satisfactory reduction in blood glucose level. However, for practical reasons, oral administration can be made more frequent than injection applications.

In one embodiment, the therapeutically effective amount of CC compound may be administered once daily. In another embodiment, the dose is administered twice or three times daily.

Considering the relatively short-term effectiveness of the CC compound, one of the recommended practical uses is to prevent large excursions in blood glucose level during a substantial meal. For this, the CC compound is taken orally or by an injection application 15-45 min prior the meal.

The CC compound may be used together with insulin or any other oral or injectable anti-diabetic medication if deemed necessary to reduce hyperglycemia and to prevent or reduce complications resulting from hyperglycemia and diabetes including increased mortality from critical illness and macrovascular as well as microvascular or other tissue injury-related events leading to cardiomyopathy, cardiovascular disease, various neuropathies, retinopathy, nephropathy, stroke, and diabetic wounds. Such diabetic medication may be chosen from sulphonylureas (e.g., Tolbutamide, Glimepiride, Glyburide, Glipizide, Tolazamide, Acetohexamine, Chlorpropamide), meglitinides (e.g., Nateglidine and Repaglinide), incretin hormones (glucagon-like peptide and glucose-dependent insulinotropic peptide as well as their analogs), inhibitors of dipeptidyl peptidase-4 (Sitagliptin), biguanides (e.g., Metformin or Glucophage), inhibitors of-glucosidase (e.g., Acarbose, Miglitol), thiazolidinediones (e.g., Pioglitazone, Rosiglitazone), Metaglip (Glipizide+Metformin), Avandamet (Rosiglitazone+Metformin), Glucovance (Glyburide+Metformin), ActoPlus (Pioglitazone+Metformin), Avandaryl (Pioglitazone+Glimepiride), Janumet (Sitagliptin+Metformin), and Duetact (Pioglitazone and Metformin), or combinations thereof. These agents are administered using the respective approved doses and administration routes while the CC compound may be administered daily or intermittently orally or by an injection method, for example at any of the dosage levels described herein.

The CC compound may also be used together with other human proteins such as alkaline phosphatase [see Z. Kiss, U.S. Pat. No. 7,014,852, “Alkaline Phosphatase to Induce Weight Loss or Reduce Weight Gain”; Z. Kiss, U.S. Pat. No. 7,048,914, “Placental Alkaline Phosphatase to Control Diabetes”, which are herein incorporated by reference in its entirety], transferrin [see Z. Kiss, U.S. patent application Ser. No. 11/616,378, “Transferrin and Transferrin-Based Compositions for Diabetes Treatment”, which is herein incorporated by reference in its entirety], or α1-acid glycoprotein [see Z. Kiss, U.S. patent application Ser. No. 11/568,926, “Alpha-1-Acid Glycoprotein for the Treatment of Diabetes,” which is herein incorporated by reference in its entirety]. Combinations of a CC compound with an alkaline phosphatase, and/or transferrin, and/or α1-acid glycoprotein are suitable to reduce hyperglycemia and to prevent or reduce complications resulting from insulin resistance, hyperglycemia and diabetes including increased mortality from critical illness and macrovascular as well as microvascular or other tissue injury-related events leading to cardiomyopathy, cardiovascular disease, various neuropathies, retinopathy, nephropathy, stroke, and diabetic wounds. When CC compound is used together with alkaline phosphatase, transferrin, or α1-acid glycoprotein, the CC compound may be administered daily or intermittently orally or by an injection method, for example at any of the dosage levels described herein. In such combinations the human proteins are administered by an injection method once, twice, or three times a week at a dose of 100-mg to 2,000-mg per m² body surface of the mammal, or at any other dosage levels described in the incorporated patents and patent applications.

The CC compounds may also be used together with other treatments, for example to treat patients with hyperglycemia or type 2 diabetes who also developed cancer or one or more other associated pathological conditions such as cachexia, sepsis, pregnancy, starvation, burn trauma, metabolic syndrome, obesity, diabetic wounds, or acromegaly.

In some embodiments, CC compounds may enhance the effectiveness of wound healing combinations [see, for example, Z. Kiss, U.S. Pat. No. 7,011,965, “Compositions and Methods for Stimulating Wound Healing and Fibroblast Proliferation”; and Z. Kiss, U.S. Pat. No. 7,312,198, “Protein Compositions for Promoting Wound Healing and Skin Regeneration”, which are incorporated herein by reference in their entirety]. For example, such treatments may comprise one or more CC compounds, an alkaline phosphatase, and optionally transferrin and/or α1-antitrypsin, and/or α1-acid glycoprotein.

In case of oral administration of the CC compound, the other treatments may be applied simultaneously or separately from the CC compound. In case of injection application, the CC compound and the other drug may be dissolved or suspended in the same physiologically compatible carrier substance, or they can be applied separately.

### EXAMPLES

### Example 1

### CCcompound1 Does Not Alter Blood Glucose Level in Normal (Non-Diabetic) Mice without Glucose Challenge

Female C57/BL/6 mice (25-27-g body weight) were deprived of food for 2 hours and then injected with 4.5-mg/kg of CCcompound1. Blood samples for glucose measurements were taken from the eyes (canthus) just before the administration of CCcompound1 (0 min) as well as 30 min and 120 min after the administration of CCcompound1. Glucose concentrations in whole blood samples were immediately measured with the Fast Glucose C test. The data are the mean±std.dev. of 5 determinations, i.e. one determination with each of the 5 animals.

The data presented in TABLE 2 show that acute treatments with CCcompound1 do not change the blood glucose level in mice that were deprived of food for 2 hours. In this experiment, the period of food deprivation was short enough that compensatory mechanisms could maintain the blood glucose level near the normal 5-mM value. The condition employed also reflects the human eating habits, because normally there is at least a 2 hours interval between meals and snacks. Thus, with normal subjects under normal conditions, CC compounds are not expected to appreciably change the physiological level of blood glucose level that is about 5-mM on average.

TABLE 2
Effect of CCcompound1 on blood glucose level in normal mice.

| Treatment with | |
|---|---|
| CCcompound1 (min) | Blood glucose (mM) |
| 0 | 5.1 ± 0.5 |
| 30 | 4.9 ± 0.4 |
| 120 | 4.9 ± 0.6 |

### Example 2

### CCcompound1 (CC1), CCcompound3 (CC3), and CCcompound19 (CC19) each Reduce Blood Glucose Level in Glucose Tolerance Test Performed with Normal Mice

C57/BL/6 female mice weighing 22-23-g and fasted for 14 hours before intraperitoneal (i.p.) administration of glucose (3-g/kg) were used. None of the animals received any food during the experiment other than glucose. The animals received i.p. injections of 4.5-mg/kg of CCcompound1, CCcompound3, or CCcompound19 either 1 hour or 24 hours prior to glucose administration as indicated in TABLE 3. Blood samples were taken from the eyes (canthus), and glucose concentrations in whole blood samples were immediately measured with the Fast Glucose C test. Each group included six animals. The data, shown in TABLE 3, are the mean±std. dev. of 6 determinations, i.e. one determination with each of the six animals. In this and all subsequent experiments the values at 0 min reflect glucose concentration in blood samples collected 1-5 minutes prior to the administration of glucose.

Treatment of mice with CCcompound1, CCcompound3, and CCcompound19 one hour prior to glucose administration (indicated in TABLE 3 as “-1 h”) reduced blood glucose levels by 33%, 19%, and 23% respectively, by the 30th min of test period (TABLE 3). The CC compounds also had glucose lowering effects at later time points. In contrast, when the treatments with CCcompound1 were performed 24 hours prior to glucose administration (indicated in TABLE 3 as “-24 h) its effects were smaller and not reproducible in additional experiments. This most probably reflects relatively rapid clearance of this compound from the circulation. Overall, this experiment demonstrated that in normal mice CC compounds have relatively rapid and substantial glucose lowering effects in the glucose tolerance test. The experiment also indicated that replacement of two methyl groups with two ethyl groups still resulted in an active compound. In addition, the position of the choline moiety did not significantly affect efficacy. Thus, it is reasonable to expect that CC compounds listed in TABLE 1 and further described in the specification also exert glucose lowering effects. Finally, this experiment indicates that CC compounds reduce blood glucose level only when it is elevated beyond the normal 4-6-mM range, thus, it is unlikely to cause hypoglycemia.

TABLE 3
CC compounds lower blood glucose in a glucose tolerance test
performed with normal mice.

| | Blood glucose (mM) | | | | |
|---|---|---|---|---|---|
| Treatment | 0 min | 30 min | 60 min | 120 min | 180 min |
| No treatment | 1.3 ± 0.2 | 8.1 ± 0.6 | 6.0 ± 0.5 | 4.5 ± 0.3 | 3.2 ± 0.3 |
| CC1; −1 h | 1.3 ± 0.3 | 5.4 ± 0.5 | 4.3 ± 0.4 | 2.8 ± 0.2 | 2.2 ± 0.2 |
| CC3; −1 h | 1.7 ± 0.5 | 6.6 ± 0.3 | 5.0 ± 0.2 | 3.6 ± 0.5 | 2.7 ± 0.4 |
| CC19; −1 h | 2.1 ± 0.5 | 5.7 ± 0.4 | 4.6 ± 0.6 | 3.1 ± 0.2 | 2.2 ± 0.4 |
| CC1; −24 h | 1.4 ± 0.2 | 7.0 ± 0.7 | 5.2 ± 0.4 | 3.4 ± 0.3 | 2.8 ± 0.2 |

### Example 3

### Concentration- and Time-Dependent Effects of CCcompound1 (CC1) on Blood Glucose in Glucose Tolerance Test Performed with Normal Mice

Female C57/BL/6 mice weighing 22-23-g and fasted for 14 hours prior to i.p. glucose administration (3-g/kg) were used. None of the animals received any food during the experiment other than glucose. The animals received i.p. injections of 2.0 or 4.5-mg/kg of CCcompound1 either 30 min, 60 min or 120 min prior to glucose administration. Blood samples were taken and glucose concentrations were immediately measured as described earlier. Each group included six animals. The data are the mean±std. dev. of 6 determinations, i.e. one determination with each of the six animals. “-min” indicates the length of period in minutes elapsed between the administration of CCcompound1 (first) and glucose (second).

The results, shown in TABLE 4, indicate that, within the margin of experimental error, CCcompound1 was about as effective at the 2-mg/kg dose as at the 4.5-mg/kg dose. Second, these doses of CCcompound1 were similarly effective whether administered 30 min or 60 min prior to glucose. However, CCcompound1 was practically ineffective when administered 120 min prior to glucose. These data indicate that an effective time for the CC compounds to be administered is around 30 min or between 30-60 min prior to meal, and it can be expected that it will remain effective for about another hour or little longer.

TABLE 4
Concentration- and time-dependent effects of CCcompound1 on blood glucose in
glucose tolerance test performed with mice.

| | Blood glucose (mM) | | | | |
|---|---|---|---|---|---|
| CCcompound1 | 0 min | 30 min | 60 min | 120 min | 180 min |
| No treatment | 1.35 ± 0.20 | 8.10 ± 0.40 | 6.10 ± 0.35 | 4.45 ± 0.35 | 3.20 ± 0.25 |
| 2 mg/kg; −30 min | 1.30 ± 0.20 | 5.40 ± 0.45 | 4.15 ± 0.45 | 3.50 ± 0.30 | 2.80 ± 0.30 |
| 4.5 mg/kg; −30 min | 1.50 ± 0.25 | 5.05 ± 0.20 | 3.55 ± 0.25 | 2.95 ± 0.25 | 1.95 ± 0.30 |
| 2 mg/kg; −60 min | 1.30 ± 0.30 | 5.70 ± 0.35 | 4.30 ± 0.30 | 3.70 ± 0.30 | 3.25 ± 0.35 |
| 4.5 mg/kg; −60 min | 1.40 ± 0.25 | 5.25 ± 0.30 | 3.90 ± 0.20 | 3.20 ± 0.15 | 2.85 ± 0.15 |
| 4.5 mg/kg; −120 min | 1.35 ± 0.15 | 7.80 ± 0.40 | 5.90 ± 0.45 | 4.30 ± 0.40 | 3.00 ± 0.20 |

### Example 4

### Combined Effects of Placental Alkaline Phosphatase (PALP) and CCcompound1 (CC1) on Blood Glucose in Glucose Tolerance Test Performed with Normal Mice

Female C57/BL/6 mice weighing 24-26-g and fasted for 12 hours before i.p. glucose administration (3 g/kg) were used. None of the animals received any food during the experiment other than glucose. One group of animals was untreated while animals in other groups received subcutaneous (s.c.) injections of 2.0 or 4.5-mg/kg of CCcompound1 either 30 min or 60 min prior to glucose administration as indicated in TABLE 5. Two groups of animals received i.p. injections of 0.4-mg per mouse (˜16-mg per kg) of highly purified PALP 24 hours prior to glucose administration. Purification of PALP was described in detail earlier [Z. Kiss, U.S. Pat. No. 7,048,914, “Placental Alkaline Phosphatase to Control Diabetes]. Animals in one of the PALP treated group also received s.c. injections of 4.5-mg/kg of CCcompound1 30 min prior to glucose administration as indicated in TABLE 5. Blood samples were taken and glucose concentrations were immediately measured as described earlier. Each group included five animals. The data are the mean±std. dev. of 5 determinations, i.e. one determination with each of the five animals. “-min” and “-h” indicates the length of period in minutes and hours, respectively, elapsed between the administration of test agents (first) and glucose (second).

The results, shown in TABLE 5, confirm that CCcompound1 at the dose of 2-mg/kg and 4.5-mg/kg doses are similarly effective. Also in this experiment, as in the previous experiment, CCcompound1 was similarly effective when administered 30 min or 60 min prior to glucose. PALP alone also significantly reduced blood glucose after 30 min of glucose administration. Furthermore, at the 30th min PALP and CCcompound1 in combination reduced blood glucose level more than either agent did alone. This indicates that combination of longer-term treatment with PALP with shorter-term treatment of CCcompound1 is a more effective method than using them alone to prevent large excursion of blood glucose level during a meal without causing hypoglycemia.

TABLE 5
Combined effects of placental alkaline phosphatase and
CCcompound1 on the blood glucose level of C57/BL/6 mice.

| | Blood glucose (mM) | | | |
|---|---|---|---|---|
| Treatment | 0 min | 30 min | 60 min | 120 min |
| No treatment | 3.6 ± 0.3 | 9.6 ± 0.5 | 6.7 ± 0.5 | 5.0 ± 0.4 |
| CC1, 2 mg/kg −30 min | 3.7 ± 0.4 | 6.6 ± 0.4* | 4.9 ± 0.6 | 4.4 ± 0.5 |
| CC1, 4.5 mg/kg | 3.6 ± 0.4 | 6.2 ± 0.4* | 4.5 ± 0.3 | 4.2 ± 0.4 |
| −30 min | | | | |
| CC1, 2 mg/kg −60 min | 3.9 ± 0.6 | 6.7 ± 0.5* | 5.2 ± 0.3 | 4.6 ± 0.3 |
| CC1, 4.5 mg/kg, | 3.8 ± 0.5 | 6.4 ± 0.3* | 4.5 ± 0.5 | 4.1 ± 0.6 |
| −60 min | | | | |
| PALP, 0.4 mg/mouse, | 3.7 ± 0.5 | 6.8 ± 0.3* | 5.3 ± 0.4 | 4.3 ± 0.5 |
| −24 h | | | | |
| CC1, 4.5 mg/kg, | 4.0 ± 0.6 | 5.2 ± 0.4** | 4.1 ± 0.5 | 4.0 ± 0.2 |
| −30 min + | | | | |
| PALP, 0.4 mg/mouse, | | | | |
| −24 h | | | | |

*Significantly (P < 0.01) different from the “No treatment” group.
**Significantly (P < 0.05) different from the group treated with 4.5-mg/kg CCcompound1 30 min prior to glucose load.

### Example 5

### Combined Effects of CCcompound1 and Insulin on Blood Glucose in Glucose Tolerance Test Performed with Normal Mice

In this experiment, a sub-optimal dose of insulin (0.02 international unit-IU) was used to probe if CCcompound1 indeed can enhance the effect of insulin on blood glucose. Again C57/BL/6 female mice weighing 22-23-g and fasted for 14 hours prior to i.p. glucose administration (3-g/kg), were used. None of the animals received any food during the experiment other than glucose. The animals received i.p. injections of 4.5-mg/kg of CCcompound1 and/or 0.02 Unit insulin 60 min and 15 min prior to glucose administration, respectively. Blood samples were taken and glucose concentrations measured as indicated earlier. Each group included six animals. The data are the mean±std. dev. of 6 determinations, i.e. one determination with each of the six animals.

As shown in TABLE 6, insulin alone had a small effect at 30 min but not at later times. As in the previous experiments, CCcompound1 alone had significant effects at all times, and at the 30 min and 60 min time points the combined effects of the two agents appeared to be additive.

TABLE 6
Combined effects of CCcompound1 and sub-optimal insulin
on blood glucose in glucose tolerance test.

| | Blood glucose (mM) | | | |
|---|---|---|---|---|
| Treatment | 0 min | 30 min | 60 min | 120 min |
| No treatment | 2.0 ± 0.4 | 8.3 ± 0.6 | 5.9 ± 0.7 | 4.2 ± 0.4 |
| Insulin | 2.1 ± 0.6 | 7.2 ± 0.4 | 5.6 ± 0.5 | 3.8 ± 0.4 |
| CCcompound1 | 1.8 ± 0.4 | 5.1 ± 0.5 | 4.1 ± 0.4 | 3.3 ± 0.3 |
| Insulin + CCcompound1 | 1.9 ± 0.3 | 3.5 ± 0.5 | 3.3 ± 0.4 | 2.9 ± 0.2 |

### Example 6

### Combined Effects of CCcompound1 and Insulin on Blood Glucose in Glucose Tolerance Test Performed with Normal Mice

In this experiment again, a sub-optimal dose of insulin was used to probe if CCcompound1 can enhance the effect of insulin on blood glucose. This time first generation hybrid BDF₁ (C57/BL/6 female×DBA/2 male) adult (10-12 weeks old) male mice weighing 28-30-g and fasted for 14 hours before i.p. glucose administration (3-g/kg) were used. These mice were about 20% heavier than mice in the previous experiment described under Example 5. None of the animals received any food during the experiment other than glucose. The animals received i.p. injections of 4.5-mg/kg of CCcompound1 and 0.02 IU of insulin 15 min and 10 min prior to glucose administration. Blood samples were taken and glucose concentrations in whole blood samples determined as described earlier. Each group included six animals. The data are the mean±std. dev. of 6 determinations, i.e. one determination with each of the six animals.

Perhaps because the body weight of mice was about 20% larger in this experiment compared to mice used in the previous experiment, this time fasting did not cause such a large drop in blood glucose level (TABLE 7). As also shown in TABLE 7, in this new experiment, insulin used at the 0.02 IU dose alone had only a small effect at 30 min and it had no effect 60 min after glucose administration. As in the previous experiments, CCcompound1 alone had significant effects at the 30 and 60 min time points, even though it was added only 15 min prior to glucose. This time, CCcompound1 and insulin had less than additive effects at the 30th min. After 60 min of glucose administration, the combined effect was equal to that of CCcompound1 alone, indicating that in animals with larger body weight the small effect of 0.02 IU insulin alone does not last beyond 30 min.

TABLE 7
Combined effects of CCcompound1 and sub-optimal amount
of insulin on blood glucose in glucose tolerance test.

| | Blood glucose (mM) | | |
|---|---|---|---|
| Treatment | 0 min | 30 min | 60 min |
| No treatment | 3.5 ± 0.4 | 8.0 ± 0.3 | 6.1 ± 0.4 |
| CCcompound1; −15 min | 3.1 ± 0.2 | 4.8 ± 0.3 | 3.5 ± 0.2 |
| Insulin; 0.02 IU; −10 min | 3.1 ± 0.4 | 6.4 ± 0.5 | 5.6 ± 0.3 |
| CCcompound1 + Insulin; 0.02 IU | 3.3 ± 0.2 | 4.2 ± 0.2 | 3.4 ± 0.2 |

### Example 7

### Effects of CCcompound1 on Insulin Tolerance as Well as Tumor Growth and Lean Body Weight Loss in the PC-3 Tumor/Cachexia Model

The experimental PC-3 human tumors were developed in a homozygous line of C.B.-171 cr scid/scid adult female mice that were kept at specified pathogen free (SPF) hygienic level. Treatments with CCcompound1 (4.5 mg/kg) were started on day 12 after tumor implantation and conducted for 5+5 days with 2 treatment-free days inserted between the 2 series of treatments. A control group (10 mice) received no treatment, while another group (10 mice) received 4.5-mg/kg of CCcompound1 once every day between days 12-24. The results, shown in TABLE 8, indicate that between days 12 and 24 after tumor implantation, the prostate tumor caused about 6.9-g loss in lean body weight (4.1-g loss in total body weight). In contrast, animals treated with CCcompound1 actually gained 6.3-g lean body weight. CCcompound1 also reduced tumor volume by nearly 50%. These data have been confirmed in another similar experiment.

Tumors and cachexia are usually associated with insulin resistance. In the above experiment, on day 25, a glucose tolerance study was performed to see if PC-3 tumors also caused insulin resistance and if chronic and/or acute treatments with CCcompound1 could reverse it. Both groups were starved for 14 hours and divided into two sub-groups; one subgroup remained untreated on day 25, another subgroup was administered 4.5-mg/kg of CCcompound1 60 min prior to administration of 3-g/kg of glucose. Blood samples were taken and blood glucose was analyzed as described earlier. The results, shown in TABLE 9, show that between 30-120 min after glucose administration, reduction of blood glucose level in animals with PC-3 tumor was slower than previously observed with animals without tumor. In contrast, in animals that received chronic (C) treatment with CCcompound1 for 12 days, reduction in blood glucose level was clearly faster. Importantly, acute (A) treatment with CCcompound1 was almost as effective in cancer mice as in normal mice in reducing blood glucose level

Since most cancer patients develop insulin resistance, the results presented in TABLE 8 and Table 9 can be extended to indicate that in cancer patients CCcompound1 will generally also improve insulin sensitivity of peripheral tissues both chronically and acutely. Since cytokines, such as tumor necrosis factor-α and interleukin 6, are common mediators of insulin resistance in many pathological conditions, a CC compound is expected to enhance insulin sensitivity in all related pathological conditions including diabetes.

TABLE 8
CCcompound1 reduces tumor weight and prevents body
weight loss in the PC-3 tumor model

| | | Days after | |
|---|---|---|---|
| | | tumor transplantation | |
| CCcompound1; mg/kg | Weight (g) | 12 | 24 |
| 0 | Total weight | 25.5 ± 0.9 | 21.6 ± 1.1 |
| | Tumor weight | 0.2 ± 0.1 | 3.1 ± 0.6 |
| | Body weight (lean) | 25.3 | 18.5 |
| 4.5 | Total weight | 25.2 ± 1.3 | 31.9 ± 2.5 |
| | Tumor weight | 0.2 ± 0.1 | 1.6 ± 0.4 |
| | Body weight (lean) | 25.0 | 30.3 |

TABLE 9
Effects of chronic (C) and acute (A) treatments with CCcompound1
on blood glucose level in the PC-3 tumor model.

| | Blood glucose (mM) | | | |
|---|---|---|---|---|
| Treatments | 0 min | 30 min | 60 min | 120 min |
| No treatment | 1.7 ± 0.5 | 9.8 ± 0.7 | 8.0 ± 0.6 | 6.5 ± 0.5 |
| CCcompound1, A | 2.3 ± 0.2 | 5.5 ± 0.6 | 4.4 ± 0.7 | 3.5 ± 0.4 |
| CCcompound1, C | 1.8 ± 0.4 | 7.1 ± 0.5 | 6.5 ± 0.4 | 4.9 ± 0.6 |
| CCcompound1, C + A | 2.1 ± 0.5 | 5.1 ± 0.4 | 3.9 ± 0.4 | 3.1 ± 0.3 |

### Example 9

### Effect of Acute Treatments of Rats with CCcompound1 or CCcompound3 on Blood Glucose in Rats

Male Wistar rats weighing 240-270-g were divided into 3 groups, each group consisting of 6 or 7 animals. To ensure standard conditions animals were starved for 14 hours before the start of the experiment. CCcompound1 and CCcompound3 were injected (i.p.) 30 min prior to injecting 3-g/kg of glucose (i.p.). Blood sugar values of the experimental animals were measured with the Fast Glucose C-test; blood samples were taken from the tail. The data are the mean±std. dev. of 6-7 determinations, i.e. one determination with each of the 6-7 animals in the respective groups.

The results presented in TABLE 10 point to a somewhat more complex action of CC compounds in rats than that observed in experiments with mice. First, in rats, starvation for 14 hours also caused hypoglycemia similar to mice. However, in rats, unlike in mice, CC compounds were able to maintain normoglycemia prior to glucose administration. According to our present knowledge, this is possible only if in the liver CC compounds are able to enhance glucagon-regulated gluconeogenesis and glycolysis and thereby increase glucose output. Third, in rats, unlike in mice, after 30 min of glucose administration CC compounds had practically no effects on blood glucose level, but after 60 min they had clear significant effects (control versus CCcompound1, P=0.004; control versus CCcompound3, P=0.054). This may reflect the time needed for a shift from stimulating glucose output to reducing glucose output and instead enhancing glucose uptake and metabolism into muscle. This may also reflect that insulin secretion as well as access to the peripheral tissues is a longer process in rats than in mice. Fourth, while at the 120th min the control animals again approached the hypoglycemic state, the CC compounds clearly prevented this process. Overall, this experiment indicates that a CC compound is capable of preventing or reducing longer-term deviations of blood glucose level in both directions.

TABLE 10
In starved rats, CC compounds exert bi-directional normalizing effects on blood
glucose level in glucose tolerance test.

| | Blood glucose (mM) | | | | |
|---|---|---|---|---|---|
| Treatment | 0 min | 30 min | 60 min | 90 min | 120 min |
| No treatment | 3.35 ± 0.31 | 9.45 ± 0.65 | 8.03 ± 0.31 | 5.70 ± 0.25 | 4.56 ± 0.28 |
| CCcompound 1 | 5.03 ± 0.44* | 9.21 ± 0.45 | 5.73 ± 0.39* | 5.11 ± 0.42 | 5.37 ± 0.36 |
| CCcompound 3 | 4.73 ± 0.24* | 9.36 ± 0.43 | 6.66 ± 0.33* | 5.71 ± 0.38 | 5.43 ± 0.60 |

*Significantly (P < 0.01) different from the values obtained in the “No treatment” group.

### Example 10

### Effects of CC Compounds on the Blood Glucose Level of OB/OB Mice.

Adult female leptin-deficient ob/ob obese diabetic inbred specified pathogen free (SPF) hygienic category mice from Charles River VRF₁ were used for these experiments. The mice weighed 32-36-g at arrival. The ob/ob obese mouse is an extensively used animal model for the study of non-insulin-dependent-diabetes mellitus (NIDDM). The mutation was propagated in the C57BL/6J (BL/6) inbred strain. Homozygous obese (ob/ob) animals developed hyperglycemia due to insulin resistance, hyperinsulinemia (to compensate for reduced insulin action), and obesity. In these animals gluconeogenesis is enhanced despite their hyperinsulinemic state.

The animals were kept in macrolon cages at 22-24° C. and 50-60% humidity, with lighting regimen of 12/12 h light dark. They had free access to tap water and were fed a sterilized standard diet (Charles River VRF₁, autoclavable). The animals were cared for according to the “Guiding Principles for the Care and Use for Animals” based upon the Helsinki declaration. The ob/ob mice gained weight rapidly and developed a marked obesity by 5-6 weeks of age. Correspondingly, food intake was greatly increased. When arrived they were 5 weeks old (when the experiment was performed with CCcompound1) or 4.5 weeks old (when the experiment was performed with CCcompound3 and CCcompound19). The treatments started 8 days later in the experiment performed with CCcompound1 and 10 days later in the experiment performed with CCcompound3 and CCcompound19.

Experimental groups selected from ob/ob mice were injected subcutaneously (s.c.) at regular intervals once daily for 14 days with CCcompound1 or an analog (CCcompound3 or CCcompound19) each at the dose of 4.5-mg per kg. Control groups were also selected from ob/ob mice that remained untreated during the entire length of the experiment. In the first experiment, for which the data is presented in TABLE 11, blood samples were taken either 45 min or 6 hours after treatments with CCcompound1. In the second experiment, for which the data is presented in TABLE 12, blood samples were taken 45 min after treatments with the analogs of CCcompound1. Blood glucose was determined with the Fast Glucose C test. Both the untreated and CC compound-treated groups included 5 animals. The data are the mean±std. dev.

In the experiment described in TABLE 11, the blood glucose concentration of untreated ob/ob mice (first column) was increased from 9.5 mM to 19.1 mM between day 1 and 14. When the blood samples were taken 6 hours after treatment with CCcompound1 (second column), there was only a slight reduction in the blood glucose level that, however, reached statistical significance (P<0.05) on day 7 and day 14. In contrast, when blood samples were taken 45 min after treatment with CCcompound1 (third column), on each day examined there was a statistically highly significant (P<0.001) drop in the blood glucose level.

These results indicated that in the ob/ob mice CCcompound1 rapidly and effectively reduces blood glucose level, however, these effects become less pronounced after 6 hours of the treatment.

Since in ob/ob mice the high blood glucose is the results of insulin resistance, the results indicate that in the short term CCcompound1 effectively overcame the result of insulin resistance, perhaps either by enhancing insulin sensitivity of peripheral tissues, or by stimulating an insulin-independent glucose uptake mechanism, or both.

TABLE 11
Time-dependent effects of CCcompound 1 on
blood glucose level in ob/ob mice

| | Glucose level (mM) | | |
|---|---|---|---|
| | | CCcompound 1 | CCcompound 1 |
| Days | No Treatment | 6 hours | 45 min |
| 1 | 9.5 ± 0.4 | 9.3 ± 0.4 | 6.2 ± 0.4** |
| 4 | 12.5 ± 0.3 | 11.3 ± 0.7 | 7.4 ± 0.3** |
| 7 | 15.0 ± 0.5 | 13.8 ± 0.4* | 8.4 ± 0.2** |
| 11 | 16.8 ± 0.6 | 15.7 ± 0.4 | 9.2 ± 0.3** |
| 14 | 19.1 ± 0.8 | 17.5 ± 0.4* | 10.2 ± 0.3** |

CCcompound 3 and CCcompound19 also significantly reduced blood glucose level in ob/ob mice when blood samples were withdrawn after treatments for 45 min; the data is shown in TABLE 12. This experiment also indicated that replacement of two methyl groups with two ethyl groups still resulted in an active compound (CCcompound3). In addition, the position of the choline moiety did not seem to significantly affect efficacy. Thus, it is reasonable to expect that other CC compounds as well listed in TABLE 1 and further described in the specification will exert rapid glucose lowering effects in obese subjects with hyperglycemia. Finally, this experiment together with the experiment shown in TABLE 11 further confirms that in diabetic mice, and by extension in diabetic human subjects, CC compounds reduce blood glucose level without causing hypoglycemia.

TABLE 12
Effects of CCcompound 3 (CC3) and CCcompound 19
(CC19) on blood glucose level of ob/ob mice

| | Blood glucose (mM) | | |
|---|---|---|---|
| Day | No Treatment | CC3 | CC19 |
| 0 | 9.2 ± 0.9 | 9.1 ± 0.6 | 9.3 ± 0.7 |
| 1 | 9.4 ± 1.0 | 7.6 ± 0.7 | 6.2 ± 0.9 |
| 5 | 12.3 ± 0.8 | 8.6 ± 0.9 | 7.7 ± 0.5 |
| 10 | 16.5 ± 1.2 | 11.0 ± 0.6 | 9.3 ± 0.9 |
| 15 | 19.5 ± 1.3 | 12.6 ± 0.5 | 10.4 ± 1.0 |

### Example 11

### Effects of CCcompound1 on Blood Glucose Level in a Glucose Tolerance Test Performed with OB/OB Mice

In this experiment, 43 days old ob/ob mice weighing 40.3±1.8-g on average were used. One group of mice (5 animals) remained untreated for the subsequent 14 days. Two other groups of mice (each including 5 animals) were treated on each day for 13 days with 4.5-mg/kg dose of CCcompound1. On the 14th day the first group received only i.p. injection of glucose (3-g/kg) (first column in TABLE 13); the second group first received s.c. injection of CCcompound1, followed six hours later by i.p. injection of glucose (3-g/kg) (second column in TABLE 13); the third group first received s.c. injection of CCcompound1, followed 45 min later by i.p. injection of glucose (3-g/kg) (third column in TABLE 13). In each case, blood was first drawn 1-5 min prior to the administration of glucose, followed by drawing blood samples at 30 min, 60 min, 120 min and 180 min. The results shown in TABLE 13 indicate that CCcompound1 can effectively lower blood glucose level even after administration of a large amount of glucose. The finding that CCcompound1 was less effective to prevent the rise in blood glucose when it was administered six hours prior to glucose (TABLE 13) is likely due to its relatively rapid clearance from the circulation. This probably means that the effect of CCcompound1 on blood glucose level requires its physical presence in the circulation and is not mediated by a secondary mechanism.

TABLE 13
Effects of CCcompound 1 on blood glucose level in a
glucose tolerance test performed with ob/ob mice.

| | Blood glucose (mM) | | |
|---|---|---|---|
| | | CCcompound 1 | CCcompound 1 |
| Time (min) | No Treatment | 6 hours | 45 min |
| 0 | 19.4 ± 0.6 | 17.8 ± 0.3* | 10.7 ± 0.2** |
| 30 | 24.0 ± 0.5 | 20.7 ± 0.2* | 11.9 ± 0.4** |
| 60 | 23.3 ± 0.5 | 20.2 ± 0.2* | 11.4 ± 0.3** |
| 120 | 22.0 ± 0.5 | 19.6 ± 0.2* | 11.0 ± 0.3** |
| 180 | 20.6 ± 0.5 | 18.9 ± 0.3* | 10.9 ± 0.3** |

*, **significantly (P < 0.01* and P < 0.001**) different from the corresponding values in the “No treatment” group.

### Example 12

### Combined Effects of CCcompound1 and Antidiabetic Human Proteins

Transferrin (TF) and α1-acid glycoprotein (AGP) are two human proteins with recently reported longer-term antidiabetic effects [Z. Kiss, U.S. patent application Ser. No. 11/616,378, “Transferrin and Transferrin-Based Compositions for Diabetes Treatment”; Z. Kiss, U.S. patent application Ser. No. 11/568,926, “Alpha-1-Acid Glycoprotein for the Treatment of Diabetes”]. In the following experiment, the possible benefit of using AGP and TF together with a CC compound to lower blood glucose level in the type 2 diabetes ob/ob model was examined.

For each experimental group, ob/ob mice were chosen (5 mice in each group) that were 43 days old and weighed on average 40.3±1.9-g on the first day of experiment. Three groups of mice were treated with s.c. injection of 4.5-mg/kg CCcompound1 once every day for 17 days. Mice in one of these CCcompound1-treated groups received no other treatment, and their blood was drawn for glucose analysis on day 14 and 17 exactly 45 min after treatment. The second CCcompound1-treated group also received 0.5-mg/mouse (˜11 mg/kg) dose of greater than 98% pure commercial human transferrin [catalogue number, T 3309 according to 2004/2005 Sigma catalogue]. In this second group the blood was drawn on day 14 just prior to TF administration and on day 17 two hours after TF and 45 min after CCcompound1 administration. An additional group of animals received only TF treatment. The third CCcompound1-treated group also received 1.2-mg/mouse (26.6-mg/kg) dose of >99% pure human AGP [catalogue number G 9885 according to 2004/2005 Sigma catalogue]; in this group the blood was drawn on day 14 just prior to AGP administration and on day 17 two hours and 45 minutes after administration of AGP and CCcompound1, respectively. An additional group of animals received only AGP treatment, and yet another group of animals received no treatment at all. On day 1, prior to any treatment, the blood glucose level of all animals was in the range of 7.2-7.9 mM.

The results shown in TABLE 14 indicate that longer-term treatments with both AGP and TF significantly lowered blood glucose level. In addition, both TF and AGP added to the shorter-term effect of CCcompound1 so that combinations of CCcompound1 with both AGP and TF were significantly greater than the effects of CCcompound1 alone. These data indicate that both AGP and TF may be used together with a CC compound, such as CCcompound1, to lower blood glucose level in diabetic subjects.

CCcompound1 and PALP were also shown in TABLE 5 to lower blood glucose level more effectively in combination than alone in a glucose tolerance test performed with non-diabetic mice.

TABLE 14
Combined effects of CCcompound 1 (CC1) and human antidiabetic
proteins on blood glucose level in ob/ob mice.

Blood glucose level (mM)

| Treatment | 14th day | 17th day |
|---|---|---|
| No treatment | 15.9 ± 0.6 | 16.1 ± 0.7 |
| CC1 | 10.9 ± 0.9* | 11.4 ± 0.4* |
| AGP | 16.0 ± 0.5 | 12.4 ± 0.6* |
| TF | 15.8 ± 0.4 | 13.6 ± 0.7* |
| CC1 + AGP | 11.4 ± 0.3* | 9.8 ± 0.3** |
| CC1 + TF | 11.3 ± 0.3* | 10.2 ± 0.4** |

*Significantly (P < 0.001) different from the corresponding values of “No treatment” group.
**Significantly (P < 0.05-0.01) different from the corresponding value obtained with CCcompound 1 alone.

1. A method to reduce abnormally high blood glucose level to within, or closer to, the normal range in humans and other mammals with insulin resistance, hyperglycemia, or diabetes, the method comprising administering a heterocyclic compound represented by the formula: 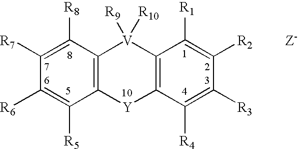
wherein R1 and R3-8 are independently hydrogen, C1-C26 straight, branched or cyclic alkanes or alkenes, aromatic hydrocarbons, alcohols, ethers, aldehydes, ketones, carboxylic acids, amines, amides, nitriles, or five- and/or six-membered heterocyclic moieties;
wherein R9 and R10 considered together are ═O or ═CH-L-N⁺(R11, R12, R13) or
wherein R9 and R10 considered independently are —OH or -L-N⁺(R11, R12, R13);
wherein R2 is represented by the formula: —X or -X′-L-N⁺(R11, R12, R13)Z⁻ or
-L-N+(R11, R12, R13)⁻;
wherein V is —S—, —Se—, —C—, —O— or —N;
wherein Y is —S—, —Se—, —C—, —O— or —N;
wherein -L-N⁺(R11, R12, R13) can be linked to V or Y if V or Y is —N or can be linked to V and Y if V and Y are both —N;
wherein X is CH3 or Hydrogen or —OH;
wherein —X′ is —CH2-, —OCH2-, —CH20-, —SCH2- or —CH2S—;
wherein L is a C1-C4 straight alkane, alkene, thiol, ether, alcohol, or amine;
wherein R11, R12 and R13 are independently Hydrogen, C1-C4 straight alkanes, alkenes, thiols, amines, ethers or alcohols; and
wherein Z- is Cl⁻, Br⁻ or I⁻.

2. The method of claim 1 wherein R11, R12, and R13 are independently methyl, ethyl, propyl, allyl, ether, sulfhydryl, amino, or hydroxyl groups.; L is —(CH₂)₂— or —(CH₂)₃—; and R₁ and R₃₋₈ are hydrogen or methyl.

3. The method of claim 1 wherein L-N⁺(R11, R12, R13) is choline.

4. The method of claim 1 wherein the compound is a thioxanthone.

5 The method of claim 4 wherein R9 and R10 considered together are ═O and R2 is —X-L-N⁺(R11, R12, R13)Z⁻.

6. The method of claim 5 wherein the compound is [3-(3,4-dimethyl-9-oxo-9H-thioxanthen-2-yloxy)-2-hydroxypropyl]trimethylammonium chloride.

7. The method of claim 5 wherein the compound is N,N,-diethyl-N-methyl-2-[9-oxo-9H-thioxanthen-2-yl)methoxy]ethanaminium iodide.

8. The method of claim 1 wherein the compound is a thioxanthene.

9. The method of claim 8 wherein R2 is O or X and R9 and R10 considered together are ═CH-L-N⁺(R11, R12, R13); L is —(CH2)2- or —(CH2)3-; and R1 and R3-8 are hydrogren or methyl.

10. The method of claim 8 wherein the compound is N,N,N-trimethyl-3-(9H-thioxanthen-9-ylidene)-propane-1-aminium iodide.

11. The method of claim 8 wherein the compound is N,N-Diethyl-N-allyl-3-(2-methyl-9H-thioxanthen-9-ylidene)-propane-1-aminium bromide.

12. The method of claim 1 wherein the heterocyclic compound is administered orally in the form of a tablet, gel capsule, or liquid, or in any other suitable form.

13. The method of claim 12 wherein the heterocyclic compound is administered orally at a dose between 100-mg to 2,000-mg per m² body surface of the mammal.

14. The method of claim 13 wherein the heterocyclic compound is administered once, twice, or thrice daily, or three-times a week, or intermittently wherein daily administration is interrupted for days or weeks.

15. The method of claim 1 wherein the heterocyclic compound is dissolved in a suitable physiologically compatible liquid carrier, such as physiological saline, and administered via an injection method selected from intravenous, intraarterial, subcutaneous, intraperitoneal, intradermal, or intramuscular, or via infusion, or by using an internally or subcutaneously inserted osmotic minipump to ensure controlled release.

16. The method of claim 15 wherein the heterocyclic compound is administered at a dose between 50-mg to 1,000-mg per m body surface of the mammal once, twice, or thrice daily, or three-times a week, or intermittently wherein daily administration is interrupted for days or weeks.

17. The method of claim 1 wherein the heterocyclic compound is administered simultaneously or sequentially with one or more oral or injectable agents suitable to reduce, prevent or treat insulin resistance, hyperglycemia, or diabetes.

18. The method of claim 17 wherein the agent or agents is/are selected from insulin, sulphonylureas (e.g., Tolbutamide, Glimepiride, Glyburide, Glipizide, Tolazamide, Acetohexamine, Chlorpropamide), meglitinides (e.g., Nateglidine and Repaglinide), incretin hormones (glucagon-like peptide and glucose-dependent insulinotropic peptide as well as their analogs), inhibitors of dipeptidyl peptidase-4 (Sitagliptin), biguanides (e.g., Metformin or Glucophage), inhibitors of α-glucosidase (e.g., Acarbose, Miglitol), thiazolidinediones (e.g., Pioglitazone, Rosiglitazone), Metaglip (Glipizide+Metformin), Avandamet (Rosiglitazone+Metformin), Glucovance (Glyburide+Metformin), ActoPlus (Pioglitazone+Metformin), Avandaryl (Pioglitazone+Glimepiride), Janumet (Sitagliptin+Metformin), and Duetact (Pioglitazone and Metformin).

19. The method of claim 17 wherein the agent or agents are administered using the respective approved doses and administration routes while the heterocyclic compound may be administered daily or intermittently orally or by an injection method at a dose of 50-mg to 2,000-mg per m² body surface of the mammal.

20. The method of claim 17 wherein the agent is alkaline phosphatase, transferrin, or α1-acid glycoprotein, or combinations of these proteins.

21. The method of claim 20 wherein the alkaline phosphatase, transferrin, or α1-acid glycoprotein are administered once, twice, or three-times a week using a dose range from 100 mg to 2,000 mg per m² body surface while the heterocyclic compound is administered daily or intermittently orally or by an injection method at a dose of 50-mg to 2,000-mg per m² body surface of the mammal.

22. The method of claim 1 wherein a heterocyclic CC compound is used in the manufacture of a composition useful for the reduction of pathologically high levels of blood glucose.
